(19) United States
(12) Patent Application Publication
(10) Pub. No.: US 2007/0156035 A1
(43) Pub. Date: Jul. 5, 2007

(54) Catheter operable to deliver IV fluids and provide blood testing capabilities

(75) Inventors: Joseph A. Graneto III, Wildwood, MO (US); James G. Gleich, St. Louis, MO (US)

Correspondence Address: PATRICK W. RASCHE;ARMSTRONG TEASDALE LLP, ONE METROPOLITAN SQUARE, SUITE 2600, ST. LOUIS, MO, 63102-2740, US

(73) Assignee: Salus Corporation d/b/a ICP Medical

(21) Appl. No.: 11/325,593

(22) Filed: Jan. 3, 2006

(51) Int. Cl. A61B 5/00

(52) U.S. Cl. 600/309; 600/573

(57) ABSTRACT

An intravenous catheter is described that includes a catheter assembly having an end portion configured for insertion into the bloodstream of a patient to deliver intravenous liquids, a valve configured to temporarily interrupt a flow of intravenous fluid into the blood stream, and an electro-chemical sampling device located near the end portion.

### BACKGROUND OF THE INVENTION

This invention relates generally to blood testing performed on patients, and more specifically to, an intravenous (IV) catheter configured to monitor patients blood serum lab values without requiring additional venapuncture, taking of blood samples, and transportation of the blood samples to a lab for testing and reporting.

Currently, nearly every patient admitted to a hospital, having outpatient surgery, or an invasive cardiology procedure requires both insertion of an IV catheter for fluid delivery and withdrawal of blood samples for blood serum lab testing. Examples of blood serum lab testing includes, but is not limited to, determining a level of electrolytes, a hemoglobin count, and a measure of hematocrit. Current procedures typically require that the patient be punctured multiple times. At least once for delivery of IV fluids and at least once more for the blood withdrawal.

Alternatively, the blood is withdrawn upon initial insertion of the IV catheter, placed in a container and sent to a lab for analysis, and once the blood samples have been withdrawn, the IV fluid delivery is initiated. However, if additional blood testing is needed, secondary venapuncture is required.

Both of these current procedures have drawbacks. One drawback is the requirement that the skin of the patient be punctured several times, once for IV fluid delivery and at least one more secondary venapuncture for blood draw. Other drawbacks include blood loss, increased likelihood of error, and the increased exposure of the healthcare provider to HIV and other blood borne pathogens with every puncture and when collecting the blood samples.

Other medical testing procedures performed at clinics, hospitals, and the like have similar drawbacks. For example, biopsies are performed, and the samples are transferred to a laboratory so that testing can be performed. Such procedures, though due care has been taken, can still result in the swapping of test results between patients.

### BRIEF DESCRIPTION OF THE INVENTION

In one aspect, an intravenous catheter is provided that comprises a catheter assembly, a valve, and an electro-chemical sampling device. The catheter assembly comprises an end portion configured for insertion into the bloodstream of a patient to deliver intravenous liquids. The valve, which in alternative embodiments may be a stop cock or flush valve, is configured to temporarily interrupt a flow of intravenous fluid into the blood stream, and the electro-chemical sampling device is located near the end portion of the catheter assembly.

In another aspect, a catheter is provided that comprises a hollow tube and an electro-chemical sampling device. The hollow tube comprises an end portion that is configured for insertion into the bloodstream of a patient. The electro-chemical sampling device is attached to the hollow tube approximate the end portion.

In still another aspect, a method for preparing an intravenous catheter for performing blood tests on a patient is provided. The method comprises configuring the catheter with an electrochemical sensing device configured for blood testing, providing a valve having an ability to interrupt any fluid infusion into the bloodstream of the patient through the catheter, and adapting the sensing device to provide data relating to blood testing to an external device.

### BRIEF DESCRIPTION OF THE DRAWINGS

FIG. 1 is a side view of a catheter that has a sampling device attached thereto, a needle extending from the catheter.

FIG. 2 is a side view of the catheter of FIG. 1, the needle removed therefrom, the end of the catheter within a blood vessel.

FIG. 3 is an illustration of an interconnection of an electro-chemical sampling device to a conductive electrode on a hub of a catheter.

FIG. 4 is a view of the catheter of FIG. 1 further illustrating a valve configured to suspend the flow of IV fluids through the catheter.

FIG. 5 is a side view of a catheter that has multiple sampling devices therein.

### DETAILED DESCRIPTION OF THE INVENTION

Described herein is a catheter that is utilized to provide, in an intravenous (IV) embodiment, IV fluid to a patient. The catheter is also configured to monitor, for example, the patient's blood serum laboratory values without requiring additional, or secondary, venapuncture. Such a catheter also eliminates the need for the taking of blood samples from the patient and also removes the need to transport the blood samples to a laboratory for testing. Such a catheter is also thought to eliminate the possibility of reporting errors as laboratory test results ate transferred from the laboratory back to the healthcare provider.

While described herein as an IV catheter, it is to be understood the embodiments may be equally applied within other medical testing applications. For example, the embodiments may be applied for urinary tract testing, angioplasty and other testing that might occur within arteries and veins, as well as a replacements for biopsies.

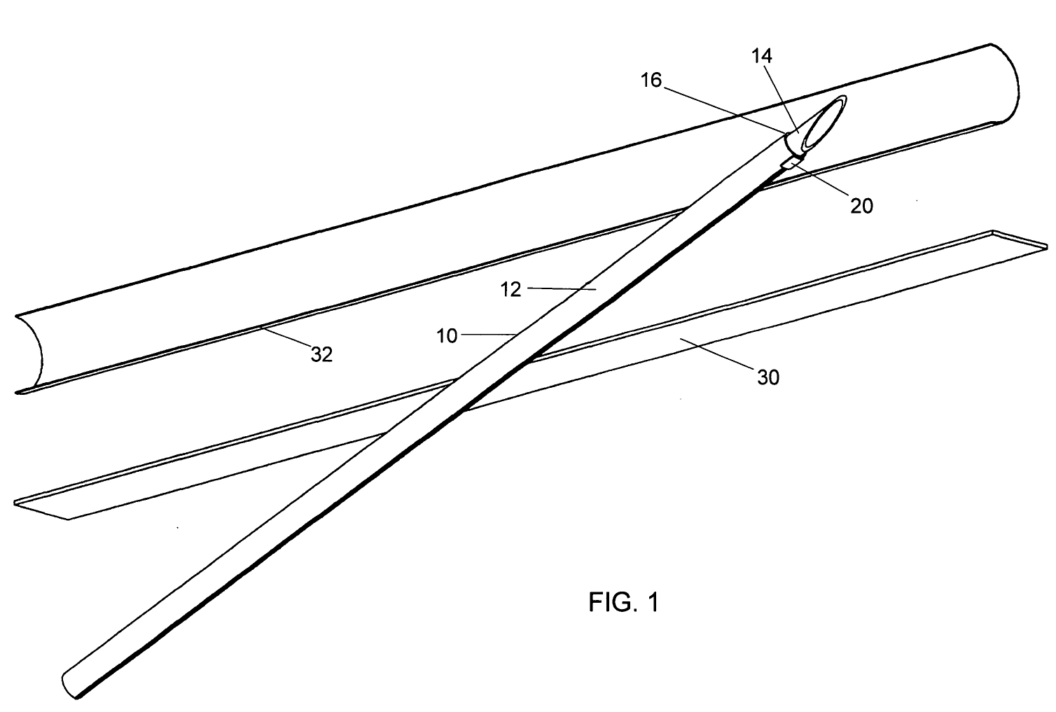

FIG. 1 is a side view illustration of an IV catheter 10 according to the present invention. Catheter 10 includes a hollow tube 12. A hypodermic needle 14 extends from an end portion 16 of hollow tube 12. An electro-chemical sampling device 20 is attached to hollow tube 12 near its end portion 16. In alternative embodiments, sampling device 20, and components associated with sampling device 20 may be attached to an interior of hollow tube 12. A conductive strip 22 is configured to extend from sampling device 20 and interface with external equipment (not shown in FIG. 1) which provides the test results to the operator.

As shown in FIG. 1, catheter 10 is configured to pierce the skin 30 of a patient and end portion 16 of hollow tube 12, including sampling device 20, is configured to be placed within the confines of a blood vessel 32 of the patient.

Catheter 10 is operable for the delivery of intravenous fluids. In addition, electro-chemical sampling device 20, through simple contact with a patients blood stream is operable to initiate lab testing sequences that can produce, for example, the patient's serum electrolyte levels, as well as hemoglobin and hematocrit testing. To provide such test results, in one embodiment, sampling device 20 is coated with one or more of glucose oxidase, dehydrogenase, and hexokinase which combine with glucose present in a bloodstream. In alternative embodiments, electro-chemical sampling device 20 may be utilized for diabetes testing as well as in applications where an electro-chemical sampling device is placed in contact with substances other than a patient's blood, for example, a urinary tract, an esophagus or stomach, and into masses that would be currently subject to biopsy. Other testing that may be performed utilizing various embodiments of sampling device 20 includes, but are not limited to, sodium level, potassium level, carbon dioxide level, venous oxygen level, calcium level, magnesium level, glucose level, BUN, platelet levels, leukocyte levels, monocyte levels, bleeding time, clotting time, and erythrocytes.

Various embodiments of sampling device 20 are reduced scale versions of sampling devices utilized in other applications. For example, similar to the test strips found in various blood glucose monitors, one embodiment of sampling device 20 is configured to perform testing based on an amount of electricity that passes through a blood sample adjacent sampling device 20. In this embodiment, the amount of electricity passing through the blood stream at sampling device 20 causes a signal to be passed through conductive strip 22 which is analyzed at the above described external equipment.

In an alternative embodiment not illustrated, catheter 10 further includes a light sourcing mechanism such that the blood sample passes between the light source and the sampling device 20. In this embodiment, the amount of light reflecting from sampling device 20 causes a signal to be passed through conductive strip 22 which is analyzed at the above described external equipment. In one embodiment, the interaction between the blood and sampling device 20 causes a color of sampling device 20 to change, which affects an amount of light that reflects from, or is absorbed by, sampling device 20.

While described herein in terms of a blood glucose monitor and a blood sample, it is to be understood that such a description should not be construed as limiting. The described embodiments of catheter 10 and sampling device 20 are applicable for performing tests outside of the intravenous application herein described, including, but not limited to, urinary tract testing, digestive tract testing, and any other application where a catheter may be utilized and for which laboratory tests are desired.

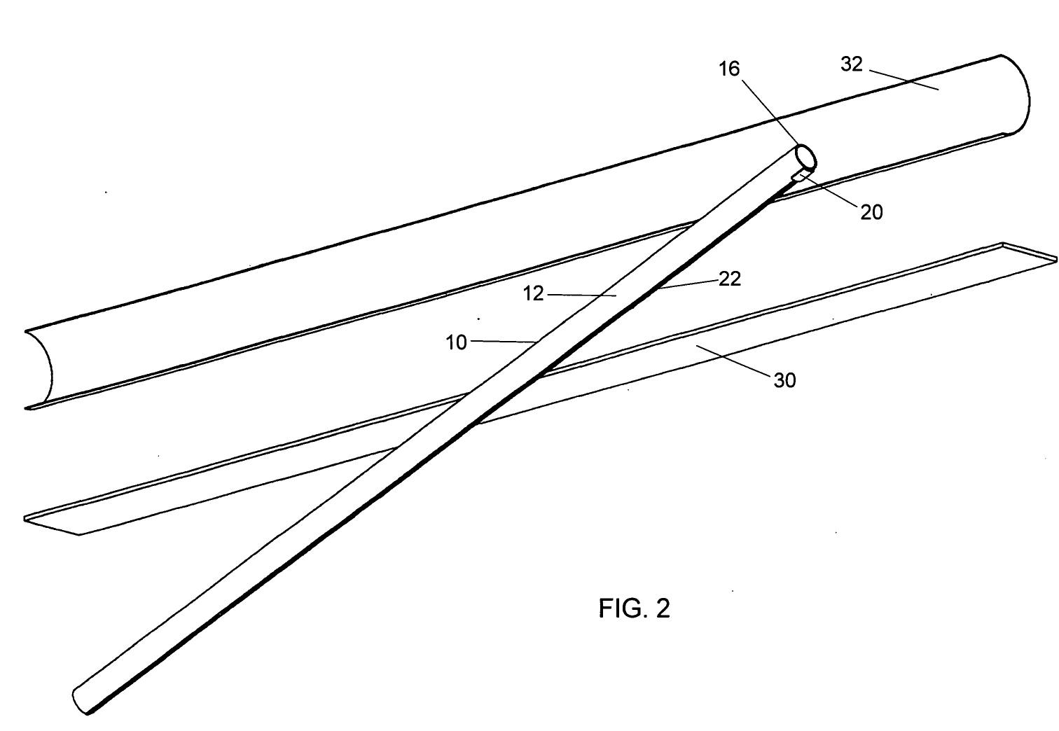

Referring once again to the IV catheter embodiment, FIG. 2 is a side view of catheter 10 with an end portion 16 within the confines of blood vessel 32. Needle 14 (shown in FIG. 1) has been extracted from hollow tube 12. IV fluids are able to pass from hollow tube 12 and into the bloodstream (artery or vein) of the patient. As further described herein, catheter 10 is operable to interrupt the flow of IV fluids into the patient. Once the flow of the IV fluids has been interrupted, electro-chemical sampling device 20 is exposed to an undiluted (by IV fluids) blood stream. At this time, device 20 is operable to provide signals that are transmitted along conductive strip 22 to external equipment (not shown in FIG. 1) which provides the blood test results to the operator.

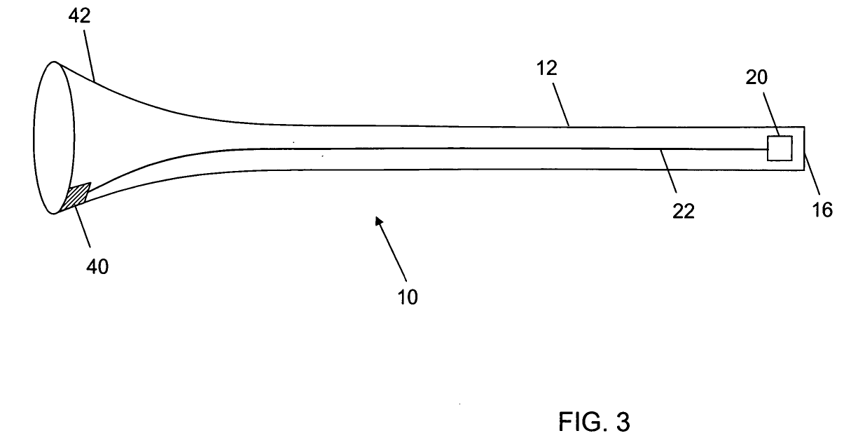

In order to produce usable test results, IV catheter 10 (and the other contemplated embodiments) include electro-chemical sampling device 20 which is configured for the particular tests to be performed. FIG. 3 is an illustration of an interconnection between electro-chemical sampling device 20 and a conductive electrode 40 on a hub 42 of catheter 10. In above described embodiments, electro-chemical sampling device 20 is a miniature or microscopic sensing strip attached to end portion 16 of hollow tube 12. In one embodiment, sampling device 20 and conductive strip 22 are attached to catheter 10 utilizing an adhesive. More specifically, a conductive electrode 40 is located along a hub 42 of catheter 10 for interconnection with the external equipment (not shown).

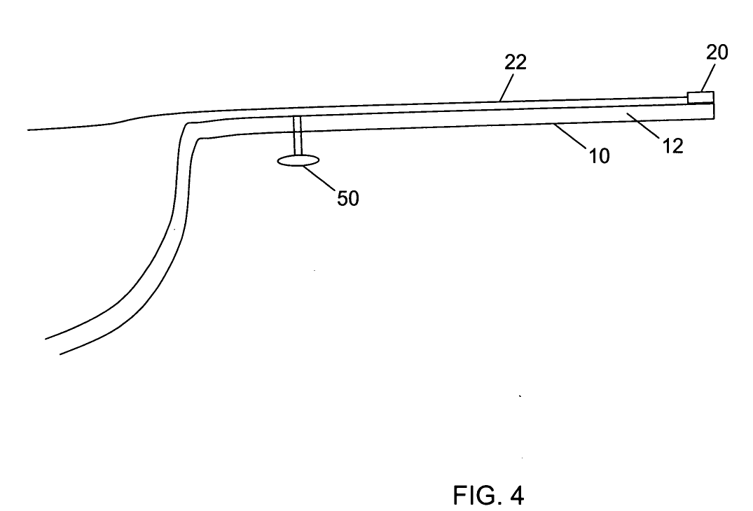

In one embodiment, to provide undiluted blood to sampling device 20, catheter 10 is configured with a valve assembly 50. FIG. 4 is an illustration of catheter 10 including valve assembly 50 incorporated therein. Valve assembly 50 allows for the temporarily interruption of IV fluid flow, allowing for non diluted blood to be in full contact with electro-chemical sensing strip 20. In alternative embodiments, valve assembly 50 is fabricated as a stop cock or a flush valve. Once the IV fluid flow has been interrupted, the care giver or other medical professional would ensure that a connection exists between conductive strip 22, and thus electrochemical sampling device 20, to an external meter or a bedside recording device that then provides immediate blood test data, for example, blood glucose level as a number. The result of utilizing catheter 10 is real time, bedside, blood test lab values, without a secondary venapuncture or blood draw.

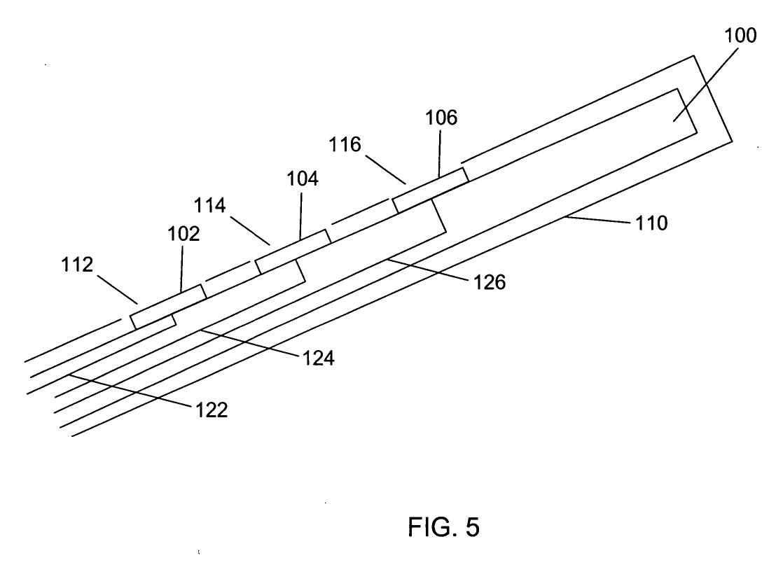

FIG. 5 is a side view of a catheter 100 including multiple sampling devices 102, 104, and 106 attached thereto. In the illustrated embodiment, a sleeve 110 substantially encircles a length of catheter 100. Sleeve 110 is further configured to be stationary with respect to catheter 100 and includes a plurality of openings 112, 114, and 116 therein which are aligned with respective sampling devices 102, 104, and 106. Such a configuration allows for multiple serum value tests to be performed utilizing a single catheter 110. In one embodiment, sampling devices 102, 104, and 106 are the same sampling device to allow for multiple values of a single test (e.g., blood glucose) to be performed. Alternatively, sampling deices 102, 104, and 106 are different sampling devices so that a battery of different lab values may be generated. Each sampling device 102, 104, and 106 is communicatively coupled to a conductive strip, 122, 124, and 126 respectively, so that test results related to each individual sampling device 102, 104, and 106 may be displayed by external equipment as described above. Utilization of sleeve 110 allows, for example, blood to contact sampling devices 102, 104, and 106 while preventing or reducing blood contact with conductive strips 122, 124, and 126. While three sampling devices 102, 104, and 106 and three corresponding openings 112, 114, and 116 are illustrated, it is to be understood that any number of each may be incorporated onto a catheter similar to catheter 100. In addition embodiments exist where multiple sampling device are “bussed” to utilize a common conductive strip.

Currently, at least some blood glucose testing for diabetics is done using a micro liter of separately drawn blood, a metering device, and an electro-chemical test strip. However, this method still requires an active puncture and a free flow of blood. Incorporating an electrochemical sampling device 20 into catheter 10 provides real time test results, eliminates blood loss, reduces error risks, and helps to eliminate the risk to the healthcare provider of exposure to HIV and other blood borne pathogens. Utilization of such a sampling device, as described above and configured for specific testing methods, also reduces the possibility of processing errors as the test results are provided at the patient, rather than from a remote lab.

While the invention has been described in terms of various specific embodiments, those skilled in the art will recognize that the invention can be practiced with modification within the spirit and scope of the claims.

What is claimed is:

1. An intravenous catheter comprising:
a catheter assembly comprising an end portion configured for insertion into the bloodstream of a patient to deliver intravenous liquids;
a valve configured to temporarily interrupt a flow of intravenous fluid into the blood stream; and
at least one electro-chemical sampling device located near said end portion.

2. An intravenous catheter according to claim 1 further comprising a conductive strip configured to communicatively couple said sampling device to an external device configured to provide medical test data based on the data received from said sampling device.

3. An intravenous catheter according to claim 1 wherein said sampling device is configured to sense contact with a bloodstream of a patient.

4. An intravenous catheter according to claim 3 wherein said sampling device is communicatively coupled to an external device, said external device configured to initiate a testing sequence when it is determined that said sampling device is in contact with an undiluted bloodstream.

5. An intravenous catheter according to claim 1 wherein said sampling device is communicatively coupled to an external device, the external device configured to calculate at least one of serum electrolytes, hemoglobin, and hematocrit, sodium level, potassium level, carbon dioxide level, venous oxygen level, calcium level, magnesium level, glucose level, BUN, platelet levels, leukocyte levels, monocyte levels, bleeding time, clotting time, and erythrocytes, based on a signal received from said sampling device.

6. An intravenous catheter according to claim 1 further comprising a conductive strip configured to communicatively couple said sampling device to an external device, said conductive strip configured to be attached along a length of said catheter assembly.

7. An intravenous catheter according to claim 1 further comprising:
a conductive electrode attached to a hub of said catheter; and
a conductive strip configured to communicatively couple said sampling device to said conductive electrode, said conductive electrode providing an interconnection to an external device.

8. An intravenous catheter according to claim 1 wherein said sampling device is configured to perform testing based on an amount of electricity that passes through a blood sample adjacent said sampling device.

9. An intravenous catheter according to claim 1 further comprising a light source, a blood sample passing between said light source and said sampling device, an interaction between the blood sample and said sampling device causing a color of said sampling device to change, thereby affecting an amount of light that reflects from or is absorbed by said sampling device.

10. An intravenous catheter according to claim 1 wherein said sampling device is coated with one or more of glucose oxidase, dehydrogenase, and hexokinase.

11. A catheter comprising:
a hollow tube comprising an end portion, said end portion configured for insertion into the bloodstream of a patient; and
an electro-chemical sampling device attached to said hollow tube approximate said end portion.

12. A catheter according to claim 11 further comprising a conductive strip configured to communicatively couple said sampling device to an external device.

13. A catheter according to claim 11 wherein said sampling device is located on an exterior of said hollow tube.

14. A catheter according to claim 11 wherein said hollow tube and said sampling device are configured to allow one or more of an optical fiber, a hypodermic needle, and a guide wire to pass through said hollow tube.

15. A catheter according to claim 11 wherein said catheter comprises one of an arterial catheter, a urinary tract catheter, and a catheter configured for biopsies.

16. A catheter according to claim 11 wherein said sampling device is configured for a specific test to be performed.

17. A catheter according to claim 16 wherein said sampling device is configured for testing at least one of serum electrolytes, hemoglobin, and hematocrit, sodium level, potassium level, carbon dioxide level, venous oxygen level, calcium level, magnesium level, glucose level, BUN, platelet levels, leukocyte levels, monocyte levels, bleeding time, clotting time, and erythrocytes.

18. A catheter according to claim 11 wherein further comprising:
a conductive electrode attached to a hub of said catheter; and
a conductive strip configured to communicatively couple said sampling device to said conductive electrode, said conductive electrode providing an interconnection to an external device.

19. A catheter according to claim 11 wherein said sampling device is configured to perform testing based on an amount of electricity that passes through a sample adjacent said sampling device.

20. A catheter according to claim 11 further comprising a light source, a sample passing between said light source and said sampling device, an interaction between the sample and said sampling device causing a color of said sampling device to change, thereby affecting an amount of light that reflects from or is absorbed by said sampling device.

21. A catheter according to claim 11 further comprising:
a plurality of said sampling devices attached to said hollow tube;
a sleeve substantially encircling a length of said hollow tube, said sleeve comprising a plurality of openings that align with respective one of said sampling devices.

22. A catheter according to claim 21 further comprising at least one conductive strip configured to communicatively couple a plurality of said sampling devices to an external device.

23. A method for preparing an intravenous-catheter for performing blood tests on a patient, said method comprising:
configuring the catheter with an electrochemical sensing device configured for blood testing;
providing a valve having an ability to interrupt any fluid infusion into the bloodstream of the patient through the catheter; and
adapting the sensing device to provide data relating to blood testing to an external device.

24. A method according to claim 23 wherein configuring the catheter with an electro-chemical sensing device comprises locating the sensing device on the catheter so as to contact a bloodstream of a patient upon insertion of the catheter.

25. A method according to claim 23 wherein adapting the sensing device to provide data relating to blood testing to an external device comprises configuring the catheter with a conductive strip that extends from the sampling device to the external device.
